(19) United States
(12) Patent Application Publication
(10) Pub. No.: US 2017/0360541 A1
(43) Pub. Date: Dec. 21, 2017

(54) EQUINE CRYOTHERAPY BOOT

(71) Applicants: Susan B. Orr, Jacksonville, FL (US); Joan C. Garibaldi, Lexington, KY (US)

(72) Inventors: Susan B. Orr, Jacksonville, FL (US); Joan C. Garibaldi, Lexington, KY (US)

(21) Appl. No.: 15/626,081

(22) Filed: Jun. 17, 2017

Related U.S. Application Data
(60) Provisional application No. 62/351,955, filed on Jun. 18, 2016.

(51) Int. Cl. A61D 7/00; A61F 7/00; A01K 13/00; A01K 29/00; A61F 7/10; A61N 5/06; A61N 2/00

(52) U.S. Cl. CPC A61D 7/00; A01K 29/005; A61F 7/0085; A61F 7/10; A01K 13/007; A61F 2007/0093; A61F 2007/0086; A61F 2007/0054; A61F 2007/0043; A61F 2007/0096; A61F 2007/108; A61N 2/002; A61N 2005/0659; A61F 2007/0056

(57) ABSTRACT

The present disclosure provides an equine therapy boot and methods of attaching the equine therapy boot to a lower extremity of a horse leg and hoof. The equine therapy boot includes an upper portion and a lower portion and an attachment means for securing the upper portion to the lower portion. The upper portion includes a fluid containment area for holding a thermal control fluid in proximity to a portion of the horse leg selected to receive treatment.

### CROSS-REFERENCE TO RELATED APPLICATIONS

This application claims the benefit of U.S. Provisional Patent Application Ser. No. 62/351,955, filed on Jun. 18, 2016, the entire content of which is hereby incorporated by reference in its entirety.

### FIELD OF THE DISCLOSURE

The present disclosure relates to apparatus and methods to effectively provide topical thermal therapy to a mid and lower leg region of an equine animal such as a horse, a mule, a donkey, a burro, a zebra, and so forth (generically, “horse”).

### BACKGROUND OF THE DISCLOSURE

Each forelimb of a horse runs from the scapula or shoulder blade to the navicular bone. In between are the humerus (arm), radius (forearm), elbow joint, ulna (elbow), carpus (knee) bones and joint, large metacarpal (cannon), small metacarpal (splint), sesamoid, fetlock joint, first phalanx (long pastern), pastern joint, second phalanx (short pastern), coffin joint, outwardly evidenced by the coronary band, and the third phalanx (coffin or pedal) bones.

Each hind limb of a horse runs from the pelvis to the navicular bone. After the pelvis come the femur (thigh), patella, stifle joint, tibia, fibula, tarsal (hock) bone and joint, large metatarsal (cannon) and small metatarsal (splint) bones. Below these, the arrangement of sesamoid and phalanx bones and joints is the same as in the forelimbs. A hoof is at a distal end of each forelimb and hind limb.

Cryotherapy, or an ice therapy, may be effective to provide at least temporary relief of swelling. Swelling is a retention of fluid that may be due to one of a number of root causes, including physical injury (e.g., a sprain or a muscle strain) or as a byproduct of an infection or other disorder. One type of disorder is endotoxemia, which is associated with acute, high levels of endotoxins in the blood that can lead to septic shock. The formation of endotoxemia may be inhibited by matrix metalloproteinases (MMPs), also known as matrixins, which are calcium-dependent zinc-containing endopeptidases.

Currently, an efficient method to provide topical ice therapy continuously to a horse, that allows the animal to be mobile in a stall, does not exist. Most equine hospital staff provide topical ice therapy to a distal limb of a horse by placing the horse's foot in a five liter intravenous fluid bag, filling the intravenous bag with ice, and taping it to the horse's fetlock with duct tape. This methodology is cumbersome, time consuming and can create sores on the horse's skin.

Cold therapy boots have been developed, but the cold therapy boots only facilitate the placement of cold packs over the hoof capsule and do not provide cooling to the lower limb region of the horse. Several forms of large ice therapy boots and baths exist that effectively cool the foot and lower limb of the horse, but these products can only be used while the horse is stationary. Accordingly, a need exists for a device to facilitate cryotherapy for a horse, while allowing the horse to have mobility in a stall.

### BRIEF DESCRIPTION OF THE DRAWINGS

The accompanying drawings, that are incorporated in and constitute a part of this specification, illustrate several embodiments of the disclosure and, together with the description, serve to explain the principles of the disclosure:

FIG. 1 illustrates multiple views of an upper portion and lower portion of an equine cryotherapy boot according to some embodiments of the present invention.

FIG. 2 illustrates an exemplary side view of an equine cryotherapy boot with chilled fluid contained within the boot.

FIG. 3 illustrates various views of exemplary embodiments of a lower portion that may be fitted to a horse's hoof.

FIG. 4 illustrates an exemplary neoprene shell that may be used in some embodiments of the present invention.

FIG. 5 illustrates an exemplary tether assembly that may be used in some embodiments of the present invention.

FIGS. 6-8 illustrate various exemplary views of securing devices that may be used in some embodiments of the present invention.

FIG. 9 illustrates an area of a horse that may be treated using an equine cryotherapy boot.

FIG. 10 illustrates exemplary method steps that may be conducted in some implementations of the present invention.

### DETAILED DESCRIPTION

Accordingly, the present invention provides a thermal containment device that may be generally described as an equine boot. The equine boot comfortably and safely provides topical thermal therapy to a horse's hoof and distal limb. Topical therapy typically will include chilled fluid, such as ice water, or ice salt water, but may also include warmed fluid. The equine boot also allows a horse receiving treatment to move freely in a stall or other area of limited movement. The design also facilitates easy and effective placement of a thermally beneficial substance, such as ice water or warm fluid or gel around the hoof and distal limb of the horse.

In general, the thermal containment device includes one or more pieces including a pliable, durable material that may be secured to a hoof wall. This could be done with various de-signs or materials, e.g., by a hook-and-loop fastener such as Velcro®, elastic straps, a grooved fastener such as Ziploc®, and so forth. This portion of the equine boot may have a zipper around a lower portion to facilitate attachment of an upper portion of the equine boot to a horse leg. The lower portion may range in a “hoof shaped” circumference of approximately 12 inches for a small boot, to approximately 20 inches for a large equine boot.

The upper portion of the equine boot may have an inner portion that will attach to the horse leg from the carpus (knee) or tarsus (hock) to the fetlock (ankle) via, for example, elastic or hook-and-loop fastener straps and so forth. The neoprene, or other water-holding or temperature-retentive material forming an outer portion of the equine boot, may be sewn or otherwise fixedly attached to the inner portion of the equine boot. A fluid-containing area formed between the inner portion and the outer portion of the equine boot may be accessible to receive and contain a thermally controlled fluid or other substance. In some embodiments, the fluid containing area may be expandable in a lower area to hold the thermally controlled fluid, such as ice and ice water. Additionally, some embodiments may include an additive to the thermally controlled fluid to keep the fluid generally microbe free and/or to provide better thermal stability.

A bottom of the outer portion may be attachable to a lower portion of the boot with a zipper, or other detachable securing device. A zipper may also be used on the outer liner to facilitate frequent placement or repositioning of the ice or other thermally controlled substance. The height of the upper portion may vary or be selectable, for example within a range of about 10-20 inches.

The bottom portion of the boot is preferably formed of a pliable, durable material and may be secured to the hoof wall. The bottom portion may be attached via, for example, hook-and-loop fastener and/or elastic straps. The bottom portion of the boot may also have a zipper around the lower portion to facilitate attachment with the upper portion of the boot. It may range in a “hoof shaped” circumference of approximately 12 inches for a small boot, to approximately 20 inches for a large boot.

The upper portion of the boot may also include an inner portion, wherein the inner portion is attachable to a horse leg from the carpus (knee) or tarsus (hock) to the fetlock (ankle) via securing devices. The securing devise may include, for example, one or both of: an elastic strap and a hook-and-loop fastener strap.

The upper portion may also include a fluid containment pouch. The fluid containment pouch may be fashioned from an outer surface material including neoprene or other fluid holding material. The outer surface material of the upper portion of the boot may be sewn or otherwise fixedly attached to an inner surface material. In preferred embodiments, the outer surface material may be expandable to hold a thermally controlled fluid, such as ice and ice water.

A bottom of the upper boot may be removably attachable to the lower portion of the boot with a zipper or other securing mechanism. A zipper or other securing mechanism also may be used to seal an opening between the inner liner and the outer liner and thereby facilitate timely placement of the ice or other thermal control substance. The height of the upper portion is resizable between about ten inches to twenty (10 to 20) inches, according to an affected area and a size of the horse receiving treatment.

In some embodiments, the boot may be zipped and unzipped to rapidly treat a horse leg, e.g., by removing water that has been warmed by the environment or body heat, and refill the boot with ice and/or chilled water.

In some embodiments, the boot may be filled with one of various fluids, such as fresh water, salt water, an alcohol, glycol (i.e., antifreeze), an oil, or various mixtures or solutions thereof. The fluid may be selected based on a desirable characteristic, such as a lower melting point, a higher rate of heat transfer, heat retention, viscosity, animal safety in case of leaks, corrosiveness, inertness, stability, and so forth.

In some embodiments, an inner portion of the boot may be wrapped tightly enough around a portion of the leg of interest (e.g., around the cannon portion) so that the boot substantially does not move, slip, slide, etc. when filled with fluid. The boot further may include an outer layer that is impermeable to unintended leakage of fluid from within the boot and infiltration of other fluids or other contaminants from outside the boot. In some embodiments, the boot further may include a replaceable liner. The liner may be provided as a layer situated between the inner portion of the boot and the leg of the animal. In other embodiments, the liner may be provided as a layer or bladder that is internal to the boot. In some embodiments, the liner may be a semi-porous material configured with microholes or the like, in order to allow fluid to leach or leak out intentionally at a slow rate, i.e., a rate at which may take several hours to drain the boot. A seeping boot may be useful to help cool at least partially a larger area of a leg that what is covered directly by the boot (e.g., by direct contact with the seepage, or by evaporation), and may be useful to make the boot lighter and more comfortable over time for the animal when worn, or to provide greater mobility and range of motion while the boot is worn.

In some embodiments, an outward-facing surface of the boot may be insulated in order to reduce heat absorbed from the environment compared to heat absorbed from the animal.

In some embodiments, the boot may include at least an outer layer made from a puncture-resistant yet relatively lightweight material, such as Kevlar® coated neoprene. In some embodiments, the outer layer may be selected from an alternate material providing a different desired quality, such as being water-retentive, flexible, stretchable, and so forth.

In some embodiments, a bottom of the boot may completely enclose the hoof. In such embodiments, the bottom of the boot (and side edges near the bottom) may be constructed to be more puncture-resistant than the top of the boot in order to resist damage caused by the hoof as it bears the weight of the animal, or from sharp edges of the hoof, or from a horseshoe nailed to the hoof, and so forth. In at least some embodiments, it is not necessary for a portion of the boot surrounding the hoof to provide cooling to the hoof.

In some embodiments, the boot may be configured as a sleeve that may surround but does not completely enclose the hoof, in particular the bottom of the hoof. Such embodiments do not need to be designed to withstand the weight of the horse, and may allow for easier draining of any seeping fluid. In some embodiments, the bottom of the sleeve may extend no lower than the top of the hoof. In other embodiments, the bottom of the sleeve may extend to the bottom of the sidewall of the hoof, which may facilitate attachment of the lower portion of the boot to the leg because the hoof may provide a relatively solid surface to attach the boot.

In some embodiments, a boot that encloses the hoof may be constructed in order to prevent or reduce ice from gathering under the hoof. For example, a predetermined amount of neoprene or the like may be used to form a water shoe with a non-slip surface underneath the hoof to shield the bottom of the hoof from ice. In some embodiments, a grating or the like may be used to allow ice to pass through the grate, while elevating the hoof above the ice.

In some embodiments, the boot may include an integrated neoprene sack or the like to hold ice, in order to provide a more targeted or intense application of cryotherapy, or to apply the cryotherapy to a hard to reach location using the rest of the boot alone. In some embodiments, a “soft” ice may be provided (i.e., having a temperature of about 32 degrees Fahrenheit (F)) or a water-ice mixture having a temperature of about 32 degrees F. to about 36 degrees F. In other embodiments, a “harder” ice may be provided (i.e., having a temperature less than 32 degrees F.). A harder ice may last longer before melting than a soft ice, but the harder ice may be too cold for the animal tissue, without additional insulation.

In some embodiments, dry ice may be used if additional insulation is provided in order to insulate the animal tissue from the temperature of dry ice (typically −109.3 degrees F.). The temperature felt by the animal tissue should be about 32 degrees F. to about 36 degrees F.

In some embodiments, cryotherapy may be applied to a relatively small localized area (e.g., to a smaller than normal hoof) by use of icepacks that are sized, contoured, or flexible to conform to the shape of a portion of the horse leg to be cooled. In some embodiments, a cloth portion may be provided to help guide a specific location for cryotherapy to be applied.

In some embodiments, the cryotherapy may be combined with a secondary therapy, e.g., an electromagnetic stimulation, an infrared therapy or a combination thereof. Embodiments may include a holder (e.g., a pocket, strap, hook and loop fastener, etc.) to secure to the boot a source for the secondary stimulation therapy. Embodiments may include an opening or the like in the boot through which to apply the stimulation.

In some embodiments, the boot may include status sensors. For example, status sensors may include a fluid level indicator, a thermometer, a pressure sensor, an electronic sensor applied to the horse leg (e.g., to measure skin temperature, pulse rate, etc.), and so forth. The sensor data may be monitored and recorded over time. A processor coupled to a memory and to the sensor may be provided, either as part of the boot, or remotely from the boot but communicatively coupled to the sensor (e.g., a wired or wireless interface). In some embodiments, the processor may be programmed by instruction code stored in the memory in order to provide a profile of treatment, either on demand or upon a regular schedule (e.g., a daily report). In some embodiments, the processor and communication interface may be configured to allow or provide remote access and/or control by a remote monitoring system or remote supervisory system. In some embodiments, the monitoring system or supervisory system may be configured to show a profile of treatment, e.g., as-provided treatment by itself, or as-provided treatment overlaid with sensor data, etc.

In some embodiments, a device to apply cryotherapy may be produced in configurations other than a boot. For example, a cryotherapy device may be fashioned in alternate configurations such as a water shoe, a bag, a knee brace, and so forth. An alternate configuration may be usable if a material it is made from includes a predetermined minimum insulation factor, and in addition may be at least partially porous and flexible (i.e., elasticity) A desired ratio of water to ice ratio may be controllable or selectable by selection of a material having a desired level of seepage. An excessive ratio of water to ice would be unnecessarily heavy and may cause sagging. In some embodiments, a ratio of ice and water of about 50% each works well. The ice / water ratio helps determine temperature of the cryotherapy device. Selecting a material with a high thermal impedance may tend to keep the cryotherapy device within a desired temperature range for a longer period of time.

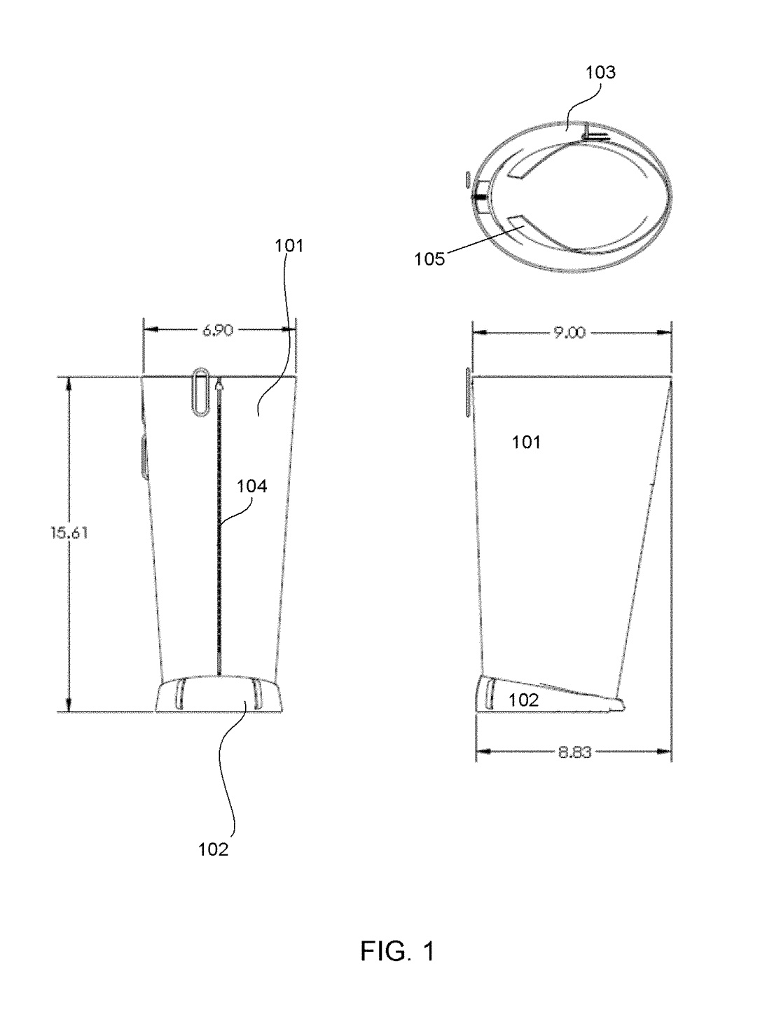

Referring now to FIG. 1, multiple views of an upper portion 101 and lower portion 102 of an equine cryotherapy boot according to some embodiments of the present invention are shown. FIG. 1 includes a bottom plan view (upper right of FIG. 1), a rear plan view (lower left of FIG. 1) and a side right plan view (lower right of FIG. 1). The equine cryotherapy boot includes a fastener 104 in a vertical dimension that may be used to secure the equine cryotherapy boot to a horse. Typically the fastener 104 may include a zipper and reside along a rear surface of the horse leg being treated. Other embodiments may include other fastener types such as hook and loop (e.g., Velcro), snaps, hook and eye or other removable fastener that fixedly secures the equine cryotherapy boot in place on the horse leg.

The lower portion 102 may include a semi-formable material, such as a neoprene, rubber, latex, plastic, PVC or other material that may provide some cushion to the horse as the horse provides pressure on the lower portion 102. The lower portion 102 may be secured in place via a strap 105 or other securing device, as illustrated in the bottom plan view. The bottom plan view also includes sidewall 103.

Although FIG. 1 and some subsequent figures include specific dimensions, other embodiments may provide the cryotherapy boot in different dimensions, including different ratios of dimensions. For example, a larger boot may be provided for larger breeds of mature horses (e.g., a Shire, a Percheron, a Clydesdale, or other draft horse, etc.), and a smaller boot may be provided for smaller breeds of horses (e.g., an Arabian, a miniature breed, etc.), or for an immature horse of any breed (e.g., a pony).

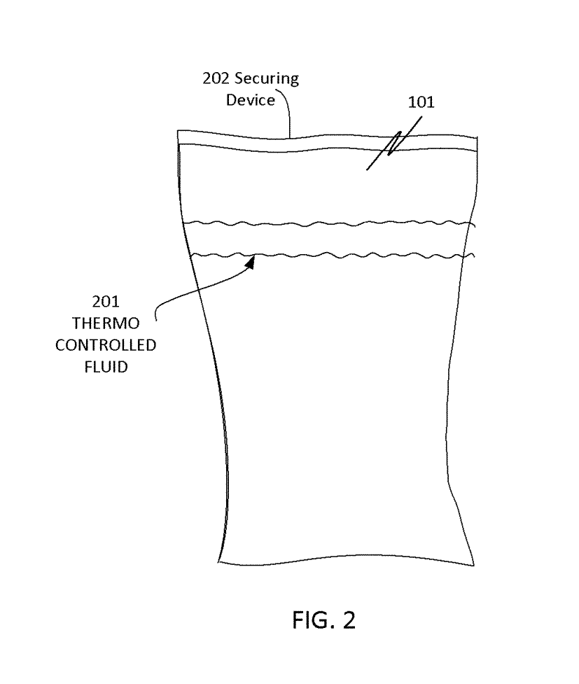

Referring now to FIG. 2, an exemplary side view of an upper portion 101 of equine cryotherapy boot is shown with thermally-controlled fluid 201 contained within the upper portion 101 of the boot. The thermally-controlled fluid typically may include chilled water, such as a mixture of ice and water, however, in some embodiments, a thermal cycling effect may be desired wherein the horse leg is first chilled and then warmed. In such embodiments, the thermally-controlled fluid may also include a warmed fluid. Temperature cycling is known to increase circulation, which may aid in healing and/or reducing inflammation. A zipper 202, or other seal may be included to close a pocket formed between an inner layer and an outer layer that contains the thermally-controlled fluid.

In some embodiments, an upper portion 101 of the boot may have an inner portion that may attach to the horse leg from the carpus (knee) or tarsus (hock) to the fetlock (ankle) via elastic and hook-and-loop fastener straps as examples. The neoprene, or other water-holding or temperature-retentive material forming an outer portion of this part of the boot, may be sewn or secured to the inner portion of this part of the boot, and may be expandable on the lower area to hold ice and ice water. The bottom of this outer liner may attach to the lower portion of the boot with a zipper. A zipper or other fastener may also be used on the outer liner to facilitate frequent placement and replacement of ice or other thermal inducing material. By way of non-limiting example, the height of the upper portion may vary from about 10 inches to about 20 inches.

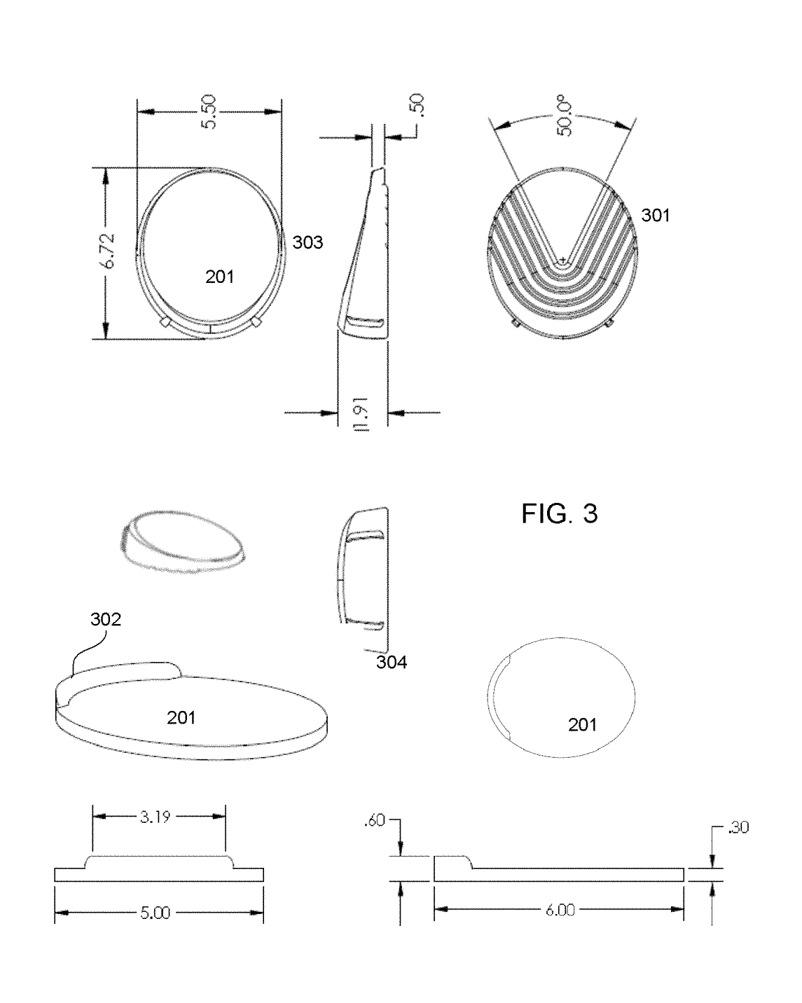

Referring now to FIG. 3, various views of exemplary embodiments of a lower portion that may be fitted to a horse hoof are illustrated. A bottom portion of the boot may include a pliable, durable material that may be secured to the lower portion of the horse leg, such as a hoof wall. Securing to the horse may be done with various designs or materials; such as, for example, via hook-and-loop fastener and elastic straps. The lower portion 201 portion of the boot may have a securing device, such as, by way of example, a zipper around the lower portion to facilitate attachment to the upper portion of the boot. Lower portion 201 may range in a “hoof shaped” circumference of approximately 12 inches for a small boot, to 20 inches for a large boot.

An upper portion of the boot may be secured to the leg of the horse via an attachment mechanism that generally corresponds to a vertical dimension of the horse leg. By way of non-limiting example, the attachment mechanism may include a zipper fixedly attached to a seam in the upper portion. The seam may run along the length of the horse leg, or cover a vertical dimension of the horse leg in another pattern, such as a spiral pattern. The spiral pattern may add strength to the overall fastening of the upper portion of the boot to the horse leg.

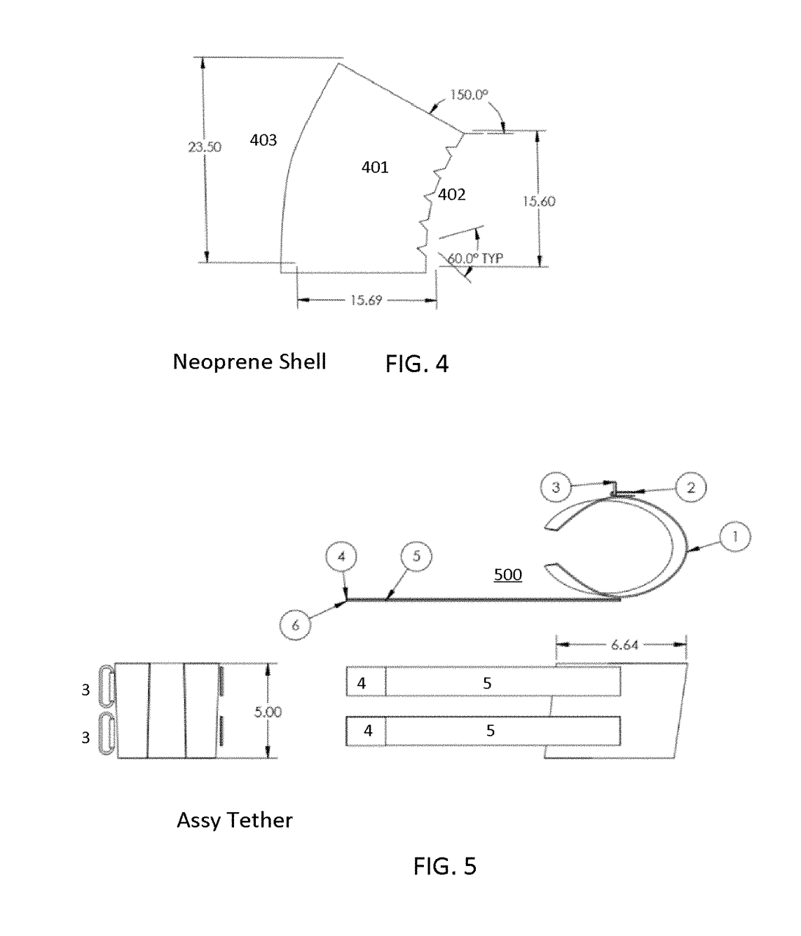

Referring now to FIG. 4, an exemplary flexible shell 401, such as a neoprene shell that may be used in some embodiments of the present invention is shown. The shell may include a side compressed from flexing 402 and a side stretched from flexing 403.

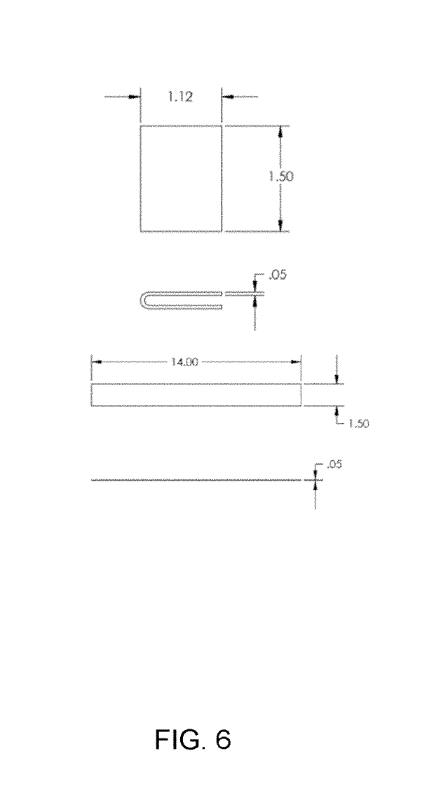

Referring now to FIG. 5, an exemplary tether assembly 500 is shown that may be used in some embodiments of the present invention. The tether assembly 500 may include an upper portion 1 with one or more straps 5. The straps 5 may interact with a buckle 3 and/or securing device 2. The straps 5 may also include a securing mechanism 4 on the end of the strap, such as hook and loop (e.g., Velcro), snaps, or hoop and eye.

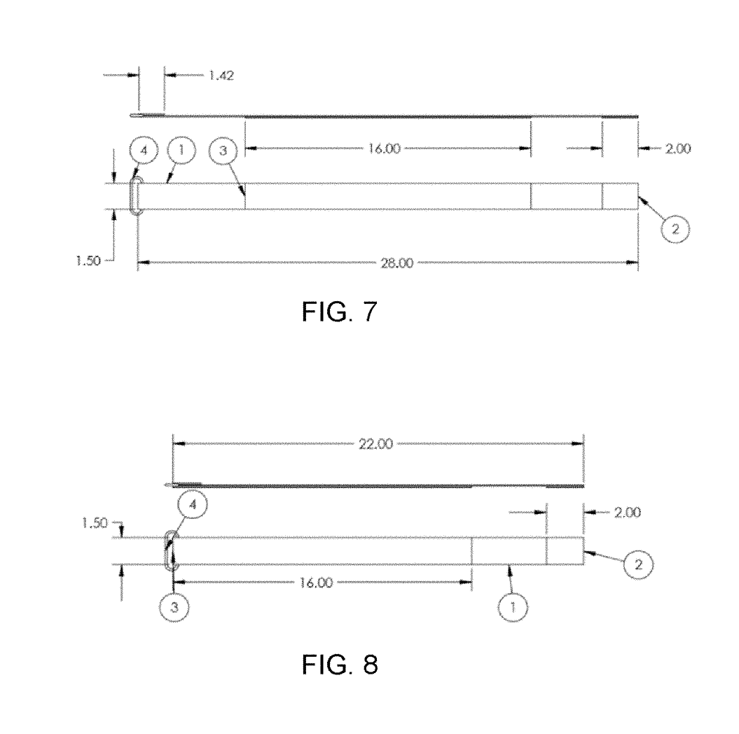

Referring now to FIGS. 6-8, various exemplary views of securing devices that may be used in some embodiments of the present invention are illustrated.

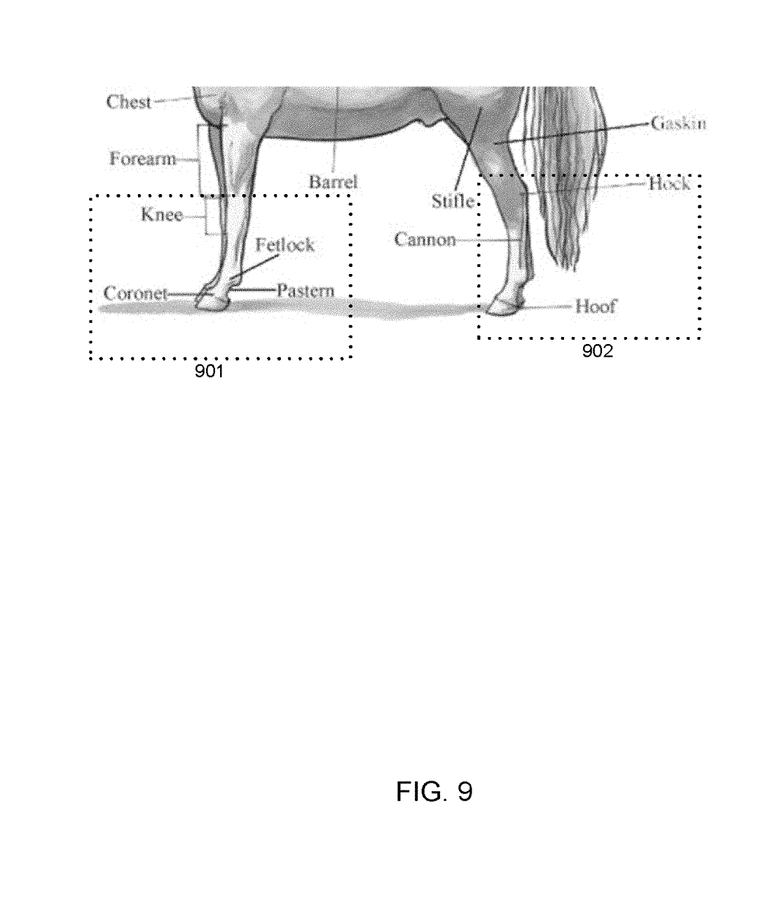

Referring now to FIG. 9, areas of a horse that may be treated using an equine cryotherapy boot are shown, and include one or both of the front legs and the back legs of the horse.

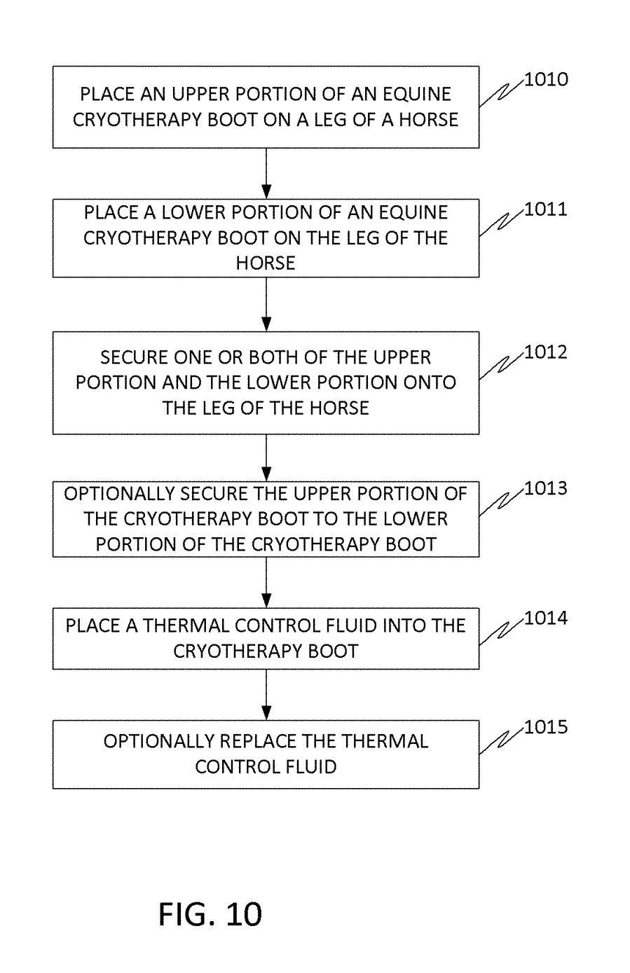

Referring now to FIG. 10, method steps that may be included in some embodiments of the present invention are listed. In an embodiment, the method may begin at step 1010, at which an upper portion of an equine cryotherapy boot is placed onto a leg of a horse.

Next, the method may progress to step 1011, at which a lower portion of the equine cryotherapy boot is placed onto a hoof of the horse.

Next, the method may progress to step 1012, at which one or both of the upper portion and the lower portion of the cryotherapy boot may be secured onto the leg of the horse.

Next, the method may progress to optional step 1013, at which the upper portion and the lower portion of the cryotherapy boot may be secured together.

Next, the method may progress to step 1014, at which a thermal control fluid may be placed into the cryotherapy boot. In other embodiments, the cryotherapy boot may be pre-filled with thermal control fluid before the cryotherapy boot is secured onto the horse leg.

Next, the method may progress to optional step 1015, at which the thermal control fluid may be replaced. For example, the fluid may be replaced if the fluid has warmed above a threshold temperature, or if more than a threshold amount of fluid has seeped from the cryotherapy boot.

These process steps are exemplary only and do not limit the scope of the invention.

### CONCLUSION

A number of embodiments of the present disclosure have been described. While this specification contains many specific implementation details, there should not be construed as limitations on the scope of any disclosures or of what may be claimed, but rather as descriptions of features specific to particular embodiments of the present disclosure.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order show, or sequential order, to achieve desirable results. In certain embodiments, multitasking and parallel processing may be advantageous. Nevertheless, it will be understood that various modifications may be made without departing from the spirit and scope of the claimed disclosure.

What is claimed is:

1. A thermal equine boot comprising:
a sleeve comprising an outer layer to encircle a lower portion of an equine leg, the sleeve comprising a fastening device along a longitudinal seam for removeably closing the seam;
a fluid chamber formed between the outer layer and the lower portion of the equine leg;
a fill port to introduce a thermal control fluid into the fluid chamber; and
a support structure to secure the thermal equine boot to the equine leg in a fashion that allows.

2. The thermal equine boot of claim 1, further comprising an inner layer coupled to an outer layer.

3. The thermal equine boot of claim 2, further comprising a lower portion to encircle a hoof of the equine leg wherein the lower portion encloses the hoof.

4. The thermal equine boot of claim 2, wherein the lower portion is detachably attached to the sleeve.

5. The thermal equine boot of claim 2, wherein the lower portion further comprises a structure to isolate the hoof from the thermal control fluid.

6. The thermal equine boot of claim 1, further comprising a semi-porous layer facing the equine leg when the thermal equine boot is secured to the equine leg.

7. The thermal equine boot of claim 6, wherein the semi-porous layer is configured to seep the thermal control fluid at a predetermined rate.

8. The thermal equine boot of claim 1, further comprising an insulated layer facing away from the equine leg when the thermal equine boot is secured to the equine leg.

9. The thermal equine boot of claim 8, wherein the insulated layer and the outer layer are integrated into a same structure.

10. The thermal equine boot of claim 1, wherein the thermal control fluid comprises an ice water mixture.

11. The thermal equine boot of claim 1, further comprising a sensor to measure a thermal condition of one of the thermal control fluid and the equine leg.

12. The thermal equine boot of claim 11, further comprising a communication interface coupled to the sensor, to report the thermal condition to a control system.

13. The thermal equine boot of claim 1, further comprising an electronic storage for storing multiple value associated with the thermal condition.

14. The thermal equine boot of claim 1, further comprising a structure to couple the thermal equine boot to a secondary stimulation source.

15. The thermal equine boot of claim 1, further comprising an additional sack to provide a cryogenic therapy to an area smaller than the size of the sleeve.

16. The thermal equine boot of claim 1, further comprising a replaceable liner situated between the inner layer and the equine leg.

17. A method of providing thermal treatment to a horse leg, the method comprising the steps of:
securing an upper portion or an equine therapy boot around a portion of a horse leg selected to receive treatment;
securing a lower portion or an equine therapy boot around a hoof corresponding with the portion of the horse leg to receive treatment;
securing the lower portion to the upper portion;
sealing a thermal control fluid in a containment area in the upper portion of the equine thermal therapy boot.

18. The method of claim 17, further comprising a step of replacing the thermal control fluid when a measured characteristic of the thermal control fluid drops below a predetermined threshold.

19. The method of claim 17, further comprising a step of allowing the thermal control fluid to seep from the containment area at a predetermined rate.

20. The method of claim 17, further comprising a step of stimulating the horse leg with a secondary stimulation.
